(19) United States
(12) Patent Application Publication
(10) Pub. No.: US 2001/0055578 A1
(43) Pub. Date: Dec. 27, 2001

(54) Scalp life conditioner

(76) Inventor: Leon Finklin, Columbia, SC (US)

Correspondence Address: E.C. Medical, P.O. Box 3101, Columbia, SC, 29230-3101, US

(21) Appl. No.: 09/850,633

(22) Filed: May 7, 2001

Related U.S. Application Data
(60) Provisional application No. 60/202,478, filed on May 8, 2000.

(51) Int. Cl. A61K 7/06

(52) U.S. Cl. 424/70.1

(57) ABSTRACT

The present invention is an improved scalp conditioner for conditions related to flaky and inflamed scalp that are commonly associated with dandruff, seborrhea, psoriasis and burns from the use of alkaline based products. The method involves administering the product to the scalp of the head and gently massaging the scalp. It can be administered several times per week.

### CROSS-REFERENCE TO RELATED APPLICATIONS

This application is a non-provisional application under 35 U.S.C. 111 (a) of its parent Provisional application Ser. No. 60/202,478, Filed May 8, 2000.

This invention is not a part of or under a government contract. The United States Government has no rights to the invention of this product.

### BACKGROUND OF THE INVENTION

The present invention is a scalp treatment composition which aids in the relief of dry scalp and inflamed and itchy areas of the scalp. This irritation is as a result of dandruff, seborrhea, psoriasis and burns from use of alkaline based hair products. The present invention assists in restoring hair loss and relieves the scalp of irritation that result from the scalp conditions previously mentioned.

Scalp conditions such as the one addressed by the present invention are dry flaky patches of dead skin, resembling dandruff, seborrhea dermatitis and psoriasis. The patches of dry skin directly associated with dandruff, seborrhea dermatosis and psoriasis usually itch. As a result of the scratching, inflammation and irritation occurs, causing trauma to the scalp, resulting in hair loss or hair breakage.

If left untreated, bacterial infection of the scalp may occur, which can penetrate to the brain causing encephalitis. Hair loss may occur as a result of the poor scalp condition. Large and loose flakes from the scalp may cause noticeable hair loss that leads to embarrassment and low self-esteem.

### PRIOR ART STATEMENT

There are several products on the market that claim relief from conditions of itchy scalp and baldness, such as dandruff, seborrhea dermatitis, psoriasis and scalp burns associated with alkaline based products. The present invention provides relief and also provides healing agents for damaged scalp. When used as instructed, it will prevent irritation of the scalp. In comparison, other products currently on the market may not provide the level of relief that Scalp Life Conditioner provides. For instance, P&S Liquid (ndc#0575400104), manufactured by Baker-Norton Pharmaceuticals is intended to help loosen and remove scales of the scalp that are associated with seborrhea. This product must be applied to the scalp every night, and shampooed out the next morning, in order to be effective. Its composition is mineral oil, glycerin, phenol, sodium chloride and salicylic acid. These ingredients are not in the present invention.

Glover's Dandruff Control Medicine (ndc#1047101143) by J.K. Pharmaceuticals Inc. consists of precipitated sulfur, salicylic acid, mineral oil and Pine oil It is intended purpose is for the relief of itching and scaling of the scalp that is associated with dandruff. It must be applied at least 2 times per week, and then thoroughly washed out. The present invention allows the product to remain on the scalp without requiring immediate shampoo.

Sulfur 8 Medicated Ointment (upc 077561043110) by J. Strickland & Company consists of sulfur, petroleum, lanolin, menthol, mineral oil and triclosan. This product is for the relief of dandruff and dry-itching, and flaking scalp associated with dandruff. Its application requires between one and four times a day, or whatever is recommended by a physician. Present invention does not require a doctor's recommendation.

Light Touch Hair & Scalp Conditioner (upc 07879814408) by Posner Laboratories, consists of mineral oil, vitamin E, lanolin, glycerin and imidazolidinyl urea. This product purpose is to help prevent hair breakage and split ends, and condition dull and dry hair and scalp. It is to be used sparingly. The ingredients in this product and the present product are not comparable.

The present invention provides flexible use for the consumer whereas, no More Flakes, (upc#632169110179) by Namaste Laboratories, LLC is intended for daily application. Its ingredients are horsetail, stinging nettle, garlic, kola nut, panthenol, castor oil, benzocaine, glycerine, menthol, soy bean oil, propylene glycol, essential fatty acids (omega 3 & omega 6) and capsicum. Unlike the herbal ingredients contained in the formula for No More Flakes, the present invention contains a different series of active ingredients that has been proven to be effective.

Scalp Cream (upc 074208500136) by Lustrasilk consists of hydrogenated vegetable oil, microcrystalline wax, cetyl alcohol, castor oil, pertolatum, lanolin and stearyl alcohol. Its purpose is to serve as a hair sheen pressing oil with very little being applied to the scalp of the hair.

The present invention aids in the relief of conditions of an irritated or inflamed scalp. It is a non-prescription product that can be purchased and easily used by the consumer. When used as described in the instructions, the results will produce a healthier scalp, promote hair growth and deter hair breakage.

### DETAILED DESCRIPTION OF THE INVENTION

The ingredients in the present invention vary in ranges from 0.25% to 3%. It is made using the following list of products.

Hydrocortisone USP (micronized)

Liquid Carbonis

Menthol

Minoxidil

Beta Carotene

Ergocalciferol

Lanolin

Paraffins

White petroleum

Hydrocortisone USP (micronized) is mixed with a prepared white petroleum, lanolin, paraffin, beta carotene and ergocalciferol base. Minoxidil is then pulverized to a fine powder and dissolved in liquid carbonis. This mixture is added to the base and mixed uniformly. Menthol is added last to produce a cool soothing affect to relieve itching. The completed product is light brown in color and has a slight sulfur-like odor. It is recommended that this product be applied directly to the scalp, massaged in well as often as two to three times a week after shampooing the hair with a mild shampoo. If irritation occurs, this product should be discontinued. It should be kept out of the reach of small children and should not be used by anyone who is sensitive to the ingredients. This product has been tested on a limited basis in several states. It has received very high marks of satisfaction from persons who have used it.

### SUMMARY OF INVENTION

This invention is a non-prescription topical scalp treatment that aids in the relief of dry and inflamed scalp conditions. It can be applied several times per week without experiencing known side effects. The product is light brown in color and has a slight sulfur like odor.

1. A non-prescription topical product for application to the scalp of the head and to the hair.

2. A product that aids in the growth of new hair.

3. A product that when applied as instructed, improves the health of the scalp.

4. A product that assists in clearing inflammation of the scalp associated with irritation from the use of alkaline base hair products.

5. A product that aids in the relief of itchy scalp associated dandruff, seborrhea and psoriasis.

6. A product that aids in clearing the scalp of crusty build up and itchy flakes.

7. A product that conditions the scalp.

8. A product that aids in the relief of dry scalp.

9. A product that can be applied 2-3 times a week without any known side effects.

10. A product that helps prevent further hair loss from the use of alkaline based hair products.

11. A product that soothes the affected areas of the scalp as it helps relieve itching.
